(19) United States
(12) Patent Application Publication
(10) Pub. No.: US 2026/0076531 A1
(43) Pub. Date: Mar. 19, 2026

(54) FLEXIBLE POWER TRANSMISSION FOR A ROBOTIC PULL ENDOSCOPE

(71) Applicant: The Regents of the University of Colorado, a body corporate, Denver, CO (US)

(72) Inventors: Mark E. Rentschler, Boulder, CO (US); Monique Reid, Boulder, CO (US)

(21) Appl. No.: 19/333,075

(22) Filed: Sep. 18, 2025

Related U.S. Application Data
(60) Provisional application No. 63/696,465, filed on Sep. 19, 2024.

(51) Int. Cl. A61B 1/00; A61B 1/04; A61B 1/045; A61B 1/12; A61B 1/31; B25J 9/10

(52) U.S. Cl. CPC A61B 1/00156; A61B 1/0004; A61B 1/00114; A61B 1/041; A61B 1/045; A61B 1/12; A61B 1/31; B25J 9/1035

(57) ABSTRACT

A self-propelled semi- or fully-autonomous robotic colonoscope device is provided that includes multi-degrees of freedom movement and may be sensor-enabled for colonoscopy procedures. The device may include a flexible rotary shaft that transmits power to a distal tip device and ultimately outputs locomotion at the distal tip with reduced complex maneuverability from the physician. In one particular implementation, a flexible metal rotary shaft may be utilized to deliver power from an off-board motor to the distal tip device. The flexible rotary shaft may be included in a tether that connects the rotary shaft from a handle to the distal tip device. The flexible shaft may be selected to provide a particular torque, directional operation, minimum bend radius, deflection, and speed. The flexible rotary shaft provides transmission of rotary motion through bends and curves of the flexible tether without sacrificing efficiency.

### CROSS-REFERENCE TO RELATED APPLICATIONS

This application is related to and claims priority under 35 U.S.C. § 119 from U.S. Provisional Application No. 63/696,465 filed Sep. 19, 2024, titled “Flexible Power Transmission for a Robotic Pull Endoscope,” the entire contents of which are fully incorporated by reference herein for all purposes.

### FIELD

Embodiments of the present invention generally relate to medical devices, and more specifically to self-propelled endoscopic devices for use in endoscopy procedures.

### BACKGROUND

Endoscopy procedures are the standard method of diagnosing and treating for a variety of lower gastrointestinal (GI) diseases, including colorectal cancers that remain the third most diagnosed and fatal cancers worldwide. Generally, these procedures are performed by manually pushing a long, semi-rigid endoscope through a three-dimensional and tortuous path colon, resulting in a technically challenging procedure. A common issue with colonoscopy procedures in particular is “looping,” where the endoscope continues to advance into the colon without a simultaneous progression of the tip, thus distending the colon and causing patient pain. Current attempts to address the challenges associated with GI procedures include active propulsion robotic capsule endoscopes (RCEs). A wide range of locomotion methods for both wireless and tethered RCEs have been attempted including legged, inchworm, treaded, magnetically linked, and even hybrid prototypes, but each category has its own drawbacks. Despite the advances made, commercially available RCEs remain problematic and unreliable for use in colonoscopy procedures.

### BRIEF DESCRIPTION OF THE DRAWINGS

FIG. 1 illustrates an isometric, three-dimensional view of a robotic capsule endoscope according to one implementation.

FIG. 2 illustrates an isometric view of a double worm drive mechanism with spur gear idling.

FIG. 3 illustrates an isometric view of a continuous track assembly threadably engaged with the double worm drive shaft to rotate a tread or track to propel the capsule robot.

FIG. 4 illustrates an overview of the robotic capsule endoscope system, including the joystick input controller, a laptop, external hardware and the robotic capsule endoscope.

FIG. 5 is a flowchart illustrating a method for controlling a capsule robotic device for an endoscopy procedure.

FIG. 6 is a diagram of a tread with inner timing-belt teeth and outer micropatterns for use with the robotic capsule endoscope disclosed herein.

FIG. 7 illustrates an isometric view of colonoscope including a distal tip with a robotic capsule, a flexible tether, and a control handle according to one implementation.

FIG. 8 illustrates an isometric view of a shaft coil for the colonoscope according to one implementation.

FIG. 9A illustrates an overhead view of the spur gear of the drive shaft transmitting power to the double worm gear of the robotic capsule endoscope disclosed herein.

FIG. 9B illustrates a front view of the spur gear of the drive shaft transmitting power to the double worm gear of the robotic capsule endoscope disclosed herein.

FIG. 9C illustrates an isometric, three-dimensional view of a drive shaft incorporating a double-worm gear and spur gear for use with the robotic capsule endoscope disclosed herein.

FIG. 10 illustrates a cross-section view of the shaft coil for the colonoscope illustrated in FIG. 7 according to one implementation.

FIG. 11 illustrates front and side views of a handle for the colonoscope illustrated in FIG. 7 according to one implementation.

### DETAILED DESCRIPTION

Presented herein is a self-propelled semi- or fully-autonomous robotic endoscope device that would address the drawbacks of other self-propelled endoscope devices discussed above. The self-propelled robotic endoscope device may provide physicians the ability to visually diagnose, biopsy, and administer therapeutics during a single procedure to diagnosis and intervene in a wide array of gastrointestinal (GI) diseases. This includes colorectal cancers (CRCs) which remain the second most common forms of cancer and the third leading cause of cancer-related deaths in the United States. Not only does the robotic capsule endoscope discussed below reduce the technical drawbacks and risks of previous endoscope devices, but the device may also reduce time, cost, and the stigma of certain endoscopic procedures, such as colonoscopies, potentially leading to increased screening rates and fewer CRC-related fatalities.

Aspects of the present disclosure involve systems, devices, methods, and the like for a self-propelled and smart-sensing robotic capsule endoscope device that reduces procedural complexity, inherent risks, patient pain, and the overall time and cost of the frequently performed endoscopy procedure, while still containing the functionality of traditional clinical endoscopes. Although discussed herein in relation to colonoscopy procedures, it should be appreciated that the robotic capsule device may be utilized in many types of endoscopic procedures, including but not limited to small intestine endoscopic procedures, bowel endoscopic procedures, or stomach endoscopic procedures. The device may provide autonomous features, such as internal navigation, internal mapping (to localize, chart progress throughout time, and to ensure a full inspection of the mucosa during a procedure), disease detection, target-tracking and disturbance rejection for therapeutic intervention (e.g., biopsy, polypectomy, etc.), autonomous intervention, and decision making (e.g., deciding when to biopsy, re-investigate certain areas, when to alert physician of issues).

In some aspects, the robotic capsule endoscope presented herein may be multi-degrees of freedom and sensor-enabled for endoscopic procedures, such as a colonoscopy procedure. The device may include a “double-worm” drive that removes axial gear forces while reducing radial moments over previous robotic endoscope devices. The full parameterization of gear geometries thereby allows for robotic capsule minimization via an optimization routine over the design constraints. The robotic device contains similar functionality of a traditional endoscope, such as a camera, adjustable LEDs, channels for insufflation and irrigation, and a tool port for common endoscopy instruments (e.g., forceps, snares, etc.). Additionally, the robotic device may include an inertial measurement unit, magnetometer, motor encoders, motor current sensors, and the like to aid in future autonomy strategies.

In another aspect, the robotic capsule endoscope may include a flexible rotary shaft that transmits power to a distal tip device. The flexible rotary shaft is, in general, a middle subassembly connecting the in vivo distal tip device and an ex vivo physician-operated handle. This flexible rotary shaft transmits power to the distal tip device and ultimately outputs locomotion at the distal tip with reduced complex maneuverability from the physician. In one particular implementation, the distal tip device may include a central drive shaft comprising a first toothed gear in mechanical communication with the flexible rotary shaft. Rotation of the flexible rotary shaft corresponds to rotation of the central drive shaft and the first toothed gear in a first rotational direction. A first parallel drive shaft comprising a second toothed gear may be mechanical communication with the first toothed gear such that rotation of the first toothed gear in the first direction corresponds to a rotation of the first parallel drive shaft in a second rotational direction. Similarly, a second parallel drive shaft comprising a third toothed gear may be in mechanical communication with the first toothed gear such that rotation of the first toothed gear in the first direction corresponds to a rotation of the second parallel drive shaft in the rotational second direction. Each of the first parallel drive shaft and the second parallel drive shaft may comprise a first screw section and a second screw section, wherein a spiraling component of the first screw section is different than a spiraling component of the second screw section to form the “double worm” gear. The rotation of first parallel drive shaft and the second parallel drive shaft is ultimately translated to the rotation of a first continuous track assembly and a rotation of a second continuous track assembly to cause maneuverability of the distal tip device.

In one particular implementation, the flexible metal rotary shaft may be utilized to deliver power from an off-board motor to the distal tip device, as described. The flexible rotary shaft may be included in a tether that connects the rotary shaft from a handle to the distal tip device. The flexible shaft may be selected to provide a particular torque, directional operation, minimum bend radius, deflection, and speed. In general, the flexible shaft may be made of layers of wires wrapped in a spiral formation. The flexible rotary shaft provides transmission of rotary motion through bends and curves of the flexible tether without sacrificing efficiency. The handle may include one or more components for operation of the distal tip device, including a joystick for automation control and one or more medical procedure components.

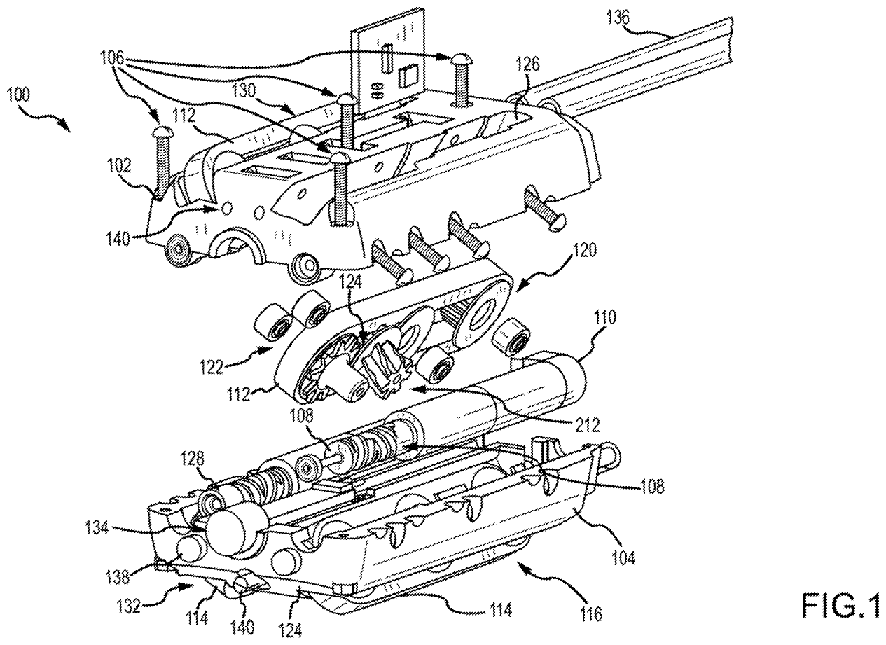

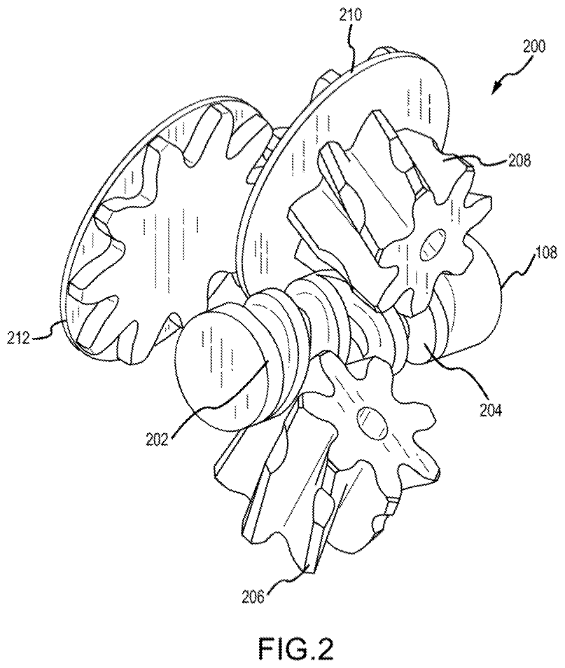

FIG. 1 illustrates an isometric, three-dimensional view of a robotic capsule endoscope (RCE) 100 according to one implementation. As shown, the robotic capsule 100 may include a housing comprising an upper portion 102 and a lower portion 104. The upper portion 102 and the lower portion 104 may be fastened together in multiple locations by the machine screws 106, which provided clamping force to hold the upper portion 102 and the lower portion 104 together and the components within the housing. A first worm support shaft 108 may be disposed within the housing between the upper portion 102 and the lower portion 104. A motor 110 may be located on an end of the first worm support shaft 108 to rotate the shaft based on one or more control signals received at the motor. In some instances, the first worm support shaft 108 may include a first screw section comprising right-handed spiraling and a second screw section comprising left-handed spiraling. For example, FIG. 2 illustrates an isometric view of a double worm drive mechanism 200 with spur gear idling. As shown in the illustration, the first worm support shaft 108 includes a first portion including right-hand spiraling 202 and a second portion including left-hand spiraling 204. Rotation of the first worm support shaft 108 causes a corresponding right-handed spiraling of the first portion 202 and a left-handed spiraling of the second portion 204.

A first worm gear 206 may be engaged with the right-handed spiraling 202 of the first worm support shaft 108. As illustrated, a counter-clockwise rotation of the first worm support shaft 108 caused by the motor 110 may cause a corresponding counter-clockwise rotation of the first worm gear 206. Similarly, a second worm gear 208 may be threadably engaged with the left-handed spiraling 204 the double worm drive shaft 108. The same counter-clockwise rotation of the first worm support shaft 108 discussed above may cause a corresponding counter-clockwise rotation of the second worm gear 208.

To achieve the overall shape of the cylindrical capsule-shaped robot 100 (i.e., longer than it is wide), the motor 110 may be parallel to the treads 112, 114 (discussed in more detail below) that are powered by the motor 110. In one implementation, a 90-degree power transmission from cylindrical DC motors 110 may be utilized. Further, to reduce gear forces and generate the smallest possible space claim, the first worm support shaft 108 is used. A single worm drive shaft can transmit power to multiple worm gears 206, 208, and thus a single motor 110 can drive the top tread 112 and bottom treads 114 in the same rolling direction. In instances where the worm gears are of the same right-handed spiraling and mirrored about the drivetrain (i.e., driving at the same angle to the surface), the worm gears produce a planar resultant force angled toward either top or bottom and a doubled axial force. However, this favoring of a top or bottom gearset and doubled axial force on the motor 110 is generally not conducive to a drivetrain of the size used in the capsule robot 100, as it necessitates additional thrust and radial bearing support. To conserve space and reduce complexity, the “double-worm” drive discussed above is used that includes both right-hand 202 and left-hand spiraling 204. With opposite handed worm gears interacting on a single screw, the radial force no longer favors top or bottom gears and instead is directed inward or outward on the mirroring plane (see FIG. 2, right). Additionally, the resultant axial force is eliminated, reducing motor strain.

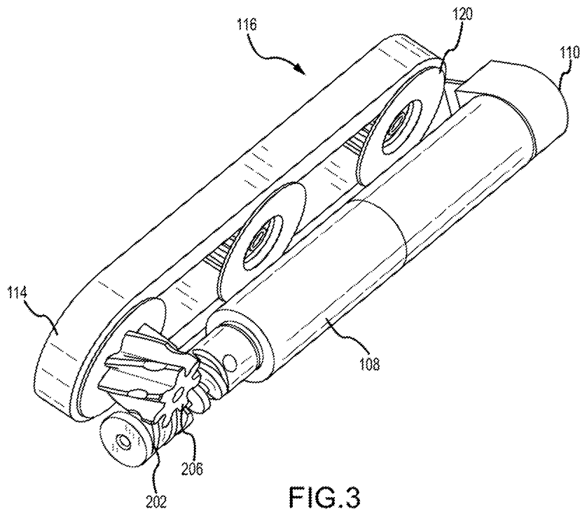

Returning to FIG. 1 and FIG. 2, the first worm gear 206 in threadably engaged with the right-handed spiraling 202 of the first worm support shaft 108 such that a counter-clockwise rotation of the first worm support shaft caused by the motor 110 may cause a corresponding counter-clockwise rotation of the first worm gear 206. In a similar manner, a clockwise rotation of the first worm support shaft 108 may cause a corresponding clockwise rotation of the first worm gear 206. The first worm gear 206 may thus be utilized to drive a rotation of a lower continuous track assembly 116. In particular, FIG. 3 illustrates an isometric view of a continuous track assembly 116 threadably engaged with the double worm drive shaft 108 to rotate a tread or track 114 to propel the capsule robot 100. The continuous track assembly 116 may include the first worm gear 206, one or more pulley gears 120, and a continuous track 114 driven by the rotation of the first worm gear 206 about the one or more pulley gears 120. For example, motor 110 may, in some instances, rotate the first worm support shaft 108 in a counter-clockwise direction about the shaft axis. Through the right-handed spiraling 202, first worm gear 206 similarly rotates in a counter-clockwise direction. Tread 114 may similarly rotate in a counter-clockwise in response to the rotation of the first worm gear 206. As shown in FIG. 1, the treads 114 may extend from the lower portion 104 of the housing through a first angled continuous track slot 124 included in the lower portion of the housing. The angled continuous track slot allow for a portion of the lower continuous track assembly 116 to extend from the lower portion 104 of the housing to engage with a surface, such as an inside surface of a GI tract. The rotation of the treads 114 of the continuous track assembly 116 in this direction may thus propel the robotic capsule in a first direction through the activation of the motor 110 and rotation of the first worm support shaft 108.

Although the double worm drive concept with opposite handed gear interactions produces favorable forces, the top treads 112 and bottom treads 114 will no longer rotate in the same rolling direction (as best seen in FIG. 2). That is, if the bottom treads 114 are rolling the device 100 forward, the top treads 112 would be attempting to roll it backward, thus causing the device to retroflex instead of propelling it in a single direction. Thus, the capsule robot 100 may include one or more idler gears 210, 212 into an upper continuous track assembly 122, thereby regaining similar rolling directions for both the lower treads 114 and the upper treads 112.

In particular and with reference to FIG. 2, an idler gear 212 may be threadably engaged with the second worm gear 208 to reverse the direction of rotation transmitted to the upper continuous track assembly 122. The idler gear 212 may engage a spur mesh of the second worm gear 208. The idler gear 212 may be engaged with an upper continuous track assembly 122 similar to the lower continuous track assembly 116 discussed above. As in a similar manner, the idler gear 212 transmits rotation of the second worm gear 208 to rotation of an upper continuous track assembly 122 in a direction opposite of the direction of the lower continuous track assembly. For example, motor 110 may, in some instances, rotate the first worm support shaft 108 in a counter-clockwise direction about the shaft axis. Through the left-handed spiraling 204 of the first worm support shaft 108, second worm gear 208 similarly rotates in a counter-clockwise direction. However, rotation of the upper continuous track assembly 122 in a counter-clockwise direction would cause the upper treads 112 to drive the capsule robotic device 100 backward, or in the opposite direction of the lower treads 114. Thus, idler gear 212 may translate the counter-clockwise rotation of the second worm gear 208 into a clockwise rotation. This clockwise rotation may be transmitted to the upper continuous track assembly 122 in a similar manner as described above. As shown in FIG. 1, the treads 112 of the upper continuous track assembly 122 may extend from the upper portion 102 of the housing through a corresponding slot 126 in the upper portion 102. The rotation of the treads 112 of the continuous track assembly 122 in this direction may, in conjunction with the rotation of the lower treads 114, propel the robotic capsule 100 in a first direction. In this manner, the double worm drive shaft 108 may drive both of the upper continuous track assembly 122 and the lower continuous track assembly 116 simultaneously while addressing the forces discussed above.

In some embodiments, the treads 112, 114 of the continuous track assemblies 116, 122 may include micropillared polydimethylsiloxane (PDMS) timing-belt style treads. The micropillared PDMS treads may be used in response to the slippery, mucosa surface upon which the capsule robotic device 100 is deployed. Further, a slight angle is provided for the continuous track assemblies 116, 122 to improve traction on the surface of the environment in which the device 100 is deployed. For example, rather than a flat planar surface that conventional vehicles encounter, the colon mucosa, although variable, is often elliptical or semi-rounded in nature. Providing the continuous track assemblies 116, 122 on a slight angle may improve the traction on such irregular, deformable surfaces.

In some embodiments, a second motor (not shown) and a second worm support shaft 128 may be included to drive a second upper continuous track assembly 130 and a second lower continuous track assembly 132. The operation and design of the second upper continuous track assembly 130 and a second lower continuous track assembly 132 may mirror that described above. Thus, the second lower continuous track assembly 132 may include a first worm gear threadably engaged with a right-hand spiraled section of the second worm support shaft 128 to transmit rotation of the second worm support shaft 128 to rotation of the second lower continuous track 132. The second lower continuous track 132 may extend through an angled slot of the lower portion 104 of the housing. Similarly, a second upper continuous track assembly 130 may include a second worm gear threadably engaged with a left-hand spiraled section of the second worm support shaft 128 to transmit rotation of the second worm support shaft 128 to rotation of the second upper continuous track assembly 130. To alter the direction of rotation of the tread 112 of the second upper continuous track assembly 130, an idler gear may be included in the assembly as explained above. The second upper continuous track 130 may extend through an angled slot of the upper portion 102 of the housing. In some embodiments, the second angled slot of the upper portion 102 of the housing may be substantially perpendicular to the first angled slot and the second angled slot of the lower portion 104 of the housing may be substantially perpendicular to the first angled slot.

The second motor and second worm support shaft 128 provides for 2-DOF locomotion through skid-steering with independently controlled left and right treads. This has the added benefits of improving the balance of the capsule robot 100 (i.e., the heaviest components of the assembly, the motors, can be placed far from the center of gravity) and allowing for an increased turning moment. Each of these motors may power treads above and below the capsule robot 100 allowing for omni-directionality may be useful in a collapsed lumen, which is the natural uninflated state of the intestine.

One or more additional features or devices may be included in or on the capsule robotic device 100 for use in colonoscopy procedures. For example, a camera device 134 may extend from a front portion of the housing for collecting video or photographs during a procedure in which the capsule robot device 100 is used. Information obtained by the camera, such as a live video feed, may be transmitted to a display device for analysis by a technician. One or more lighting devices 138, such as a light-emitting diode (LED), may be included in the device 100 to provide lighting for video and/or photography by the camera 134.

In one example, a flexible tether may extend from the rear of the device 100 and house any number and type of wires, tubes, or other transporting media for use by the robotic device 100. The wires housed in the tether may be connected to a video processing device and/or display for use by a technician during a colonoscopy procedure. In another example, the tether may house one or more motor control wires for transmitting control or activation signals to the motors 110 of the capsule device 100. Through transmission of control signals to the motor 110 along the tether, the movement of the capsule robotic device 100 may be controlled. As described in greater detail below, the tether may also include a flexible drive shaft for rotation of the one or more double worm drive shafts 108 of the capsule robotic device 100.

As noted, the tether may include an air tube, a water tube, or a combination of air and water tubing 136. The air/water tubing 136 may transport air (for inflation of an intestine) and/or water (for cleansing of an area) during a procedure. One or more air/water channels/spouts 140 may be located on the front of the device 100 to provide for delivery of the air/water carried on the air/water tubing 136. A tool port 140 may also be disposed on the front portion of the device 100 for activation of one or more tools associated with a colonoscopy procedure. For example, a forceps tool may be integrated with the tool port 140 for collecting biopsies during a procedure. The forceps may be controlled through the tool port 140, which may include a control line housed within the tether trailing the robotic device 100. Control signals transmitted on the control line may activate one or more tools integrated with the tool port 140. Other tools may also be used with the device 100, including snares, tattoo needles, and the like). Additional autonomous features, such as colon navigation, internal mapping (to localize, chart progress throughout time, and to ensure a full inspection of the mucosa during a procedure), disease detection, target-tracking and disturbance rejection for therapeutic intervention (e.g., biopsy, polypectomy, etc.), autonomous intervention, and decision making (e.g., deciding when to biopsy, re-investigate certain areas, when to alert physician of issues) may also be implemented through control of the capsule robotic device 100.

Additional surgical tools, features, and sensing hardware could be fit into gaps within the housing of the capsule robotic device 100. For example, vision, lighting, insufflation channel, water channel, and a channel for multiple endoscopic tools (e.g., biopsy forceps, snares, tattoo needles, etc.) may all be incorporated into the housing of the device 100. Additionally, the capsule robotic device 100 may incorporate electronic sensing capabilities. For example, a progressive-scanning CMOS camera may be modified from an off-the-shelf otoscope by modifying the lens/housing to allow for a more appropriate focal length, approximately 3 inches in front of the robot 100. This USB-connected camera may enable the setting of a manual exposure time and fixed frame rate, facilitating future image processing. For inertial and pose sensing, a 6-DOF inertial measurement unit (IMU) with a 3-DOF magnetometer may be designed to fit within the device 100. To better observe and control the motors 110, dual channel encoders may be present onboard the device's two motors 110, and an octal current/voltage sensor may be implemented externally on a printed circuit board (PCB) to read current draw from each motor over a connection.

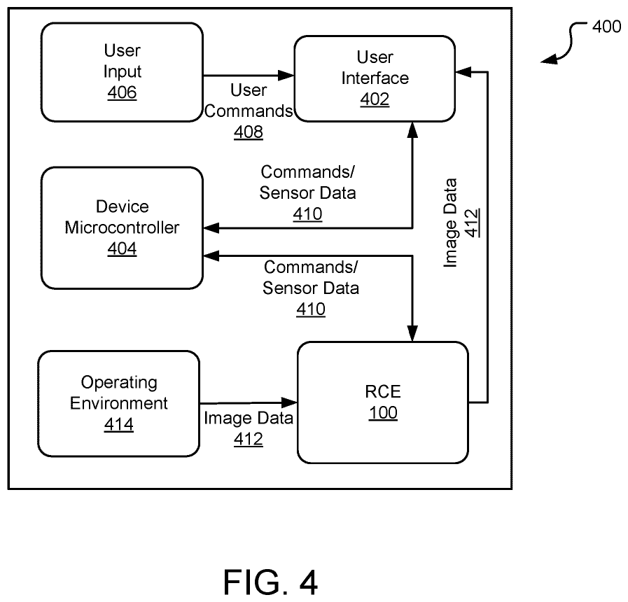

Aspects of the present disclosure may be utilized in conjunction with an RCE device 100 and control system of the RCE device. In one implementation, a small camera 134 and one or more LEDs 138 may be housed in the front of the device. The RCE 100 may also incorporates tool port 140 for biopsy forceps/snares, irrigation and insufflation. The RCE 100 and the additional system components are shown in FIG. 4. The RCE 100 may be similar to that described above with relation to FIGS. 1-3. In one implementation, the RCE 100 may be controlled externally via a user interface 402 via a wired or wireless device in communication with an onboard or separate microcontroller 404. For example, the RCE 100 may be controlled through a handle connected to the RCE through a tether, as described in more detail below. During control of the RCE 100, one or more user commands 408 may be received as user input 406 and provided to the user interface 402. The user interface 402 may communicate with the microcontroller 404 to provide one or more of the received commands or one or more generated control commands. The microcontroller 404 may process and/or transmit the commands 410 to the device 100 to control the device operations. Further, image data 412 may be obtained from the operating environment 414 by the RCE 100, such as through a camera or any other sensor of the RCE. The image data 412 may then be transmitted to the user interface 402 for display on a display device. Sensor data 410 may also be obtained by the RCE 100 from the operating environment 414 and provide to the device microcontroller 404 that may process the sensor data 410 or provide the sensor data to the user interface 402 for display. These and other operations of the RCE 100 are described in greater detail herein.

In some instances, a custom offboard PCB may be used to control the device 100. The custom PCB may contain most of the necessary hardware for controlling the device, including the onboard processing and communications unit for the device 100. The PCB may utilize a microcontroller to send and interpret digital and analog input/output signals. Commands and data to/from the device 100 may be sent via a serial connection to/from a communications interface. As described in more detail below, the custom PCB may be located within or otherwise in communication with a handle device through which user inputs may be given and used to control the RCE 100.

In some implementations, the camera and microcontroller 404 may both connect via USB to a computing device and a simple graphical user interface (GUI) 402 may provide a way to both send commands and display/collect data over the serial port. User inputs 406 may control motor speed, LED brightness, and to set device modes and data collection. Onboard data from the capsule robotic device 100, including an encoder, inertial measurement unit (IMU), current and camera data may all be collected and displayed and saved to file via the GUI, while control commands may be passed to the device 100 while also being saved to the data file.

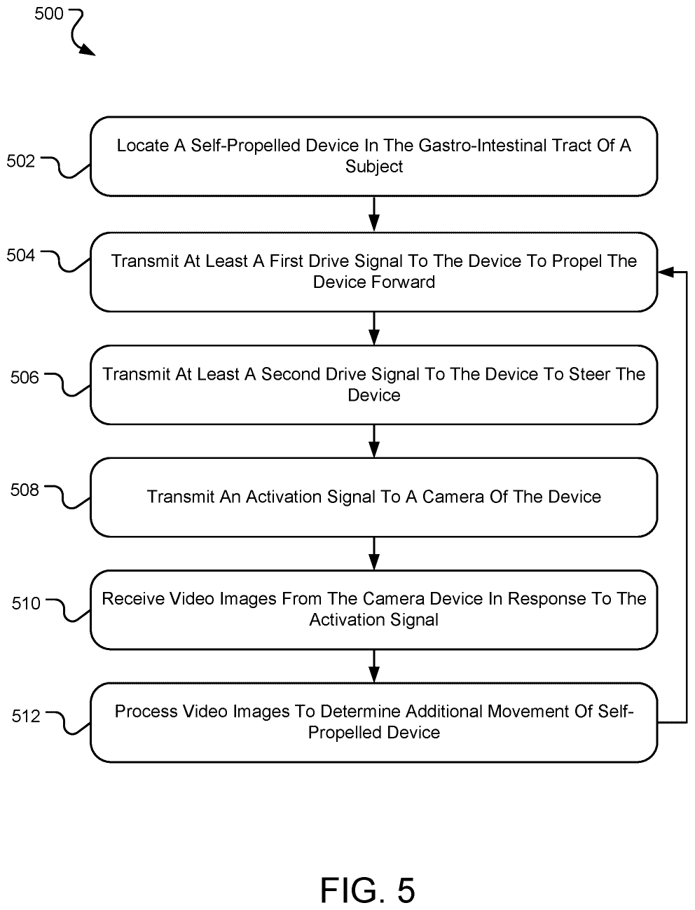

FIG. 5 is a flowchart illustrating a method 500 for controlling a capsule robotic device for a colonoscopy procedure. The method 500 may be performed by one or more technicians, physicians, computing devices, and the like to perform such a procedure as a colonoscopy. As such, one or more of the operations of the method 500 may be performed manually by the physician or through providing inputs to a computing device to control a capsule robotic device 100, such as the device described herein.

Beginning in operation 502, a self-propelled robotic device 100 may be located in the gastro-intestinal tract of a subject. In one example, the device 100 may be located in the gastro-intestinal tract of a subject to perform or during a colonoscopy procedure. In operation 504, a computing device may be utilized to transmit at least one first drive signal to the device to propel the device forward within the GI tract. In some instances, a second drive signal may be transmitted to the robotic device 100 in operation 506 to aid in the forward propulsion of the self-propelled device.

In operation 508, a camera activation signal may be transmitted to the device to begin collecting images from the camera. In some instances, a tether extending from the rear of the device 100 may house an electrical wire through which the activation signal is transmitted to activate the camera. In other instances, the activation signal may be generated onboard the robotic device 100 from a CPU located or adjacent the device. For example, the robotic device 100 may include a CPU or other processing device to perform navigation decisions, processing decisions, activation decisions, and the like. In addition, the captured images may be provided to a computing device or display device in operation 510. The captured images may similarly be transmitted via a wireless connection or via one or more conducting wires of the tether. In a similar manner, other controllable elements of the device 100 may be controlled through one or more activation signals transmitted to the device. For example, one or more lights, other sensors, collecting devices, tools, etc. may be activated through a control signal transmitted to the device. In some instances, the activation signal may activate an air or water spout to deliver air or water into the GI tract.

In operation 512, the video images, or any feedback information from the robotic capsule device 100, may be processed to determine additional movements of the device. For example, the video images may indicate that an obstruction is in front of the device 100. The device 100, an operator of the device, a controller or computing device in communication with the device, or any other processing system may determine the device should be turned in one direction or another based on the obstruction in front of the device. Processing of the images may be accomplished through image processing by a computing device, the robotic device, or through an administrator/physician controlling the device. Based on the processing of the images, additional drive signals may be transmitted to the device 100, as indicated in the flowchart 500 returning to operation 504 to repeat the above operations.

The continuous timing belt tread assembly of the robotic device 100 may provide benefits over previous devices. For example, a timing-belt is used to transfer power from the front to rear wheels/pulleys. Micropattern treads may be disposed on the outside of the tread to gain traction on the slippery colon mucosa. A process to combine inner timing-belt teeth with outer micropatterns may thus be used to create the treads from material having tunable elasticity for micropattern performance.

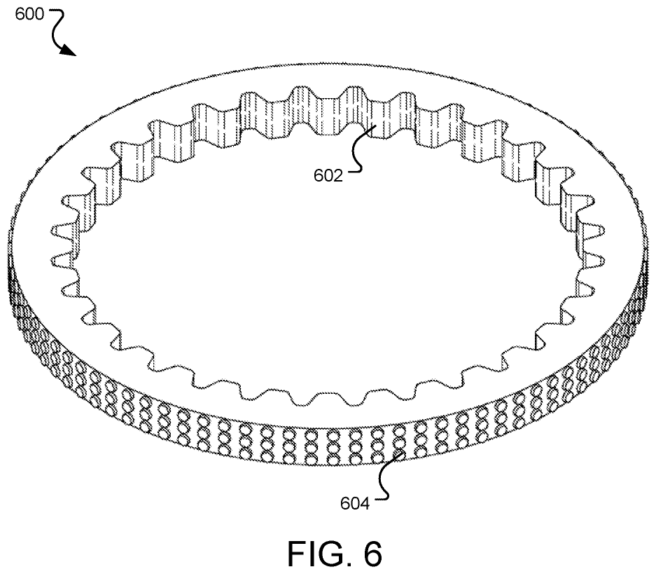

The molding process provides a continuous, single material elastic belt with timing-belt teeth on the inside (to grip the pulleys/wheels and transfer power from front to back) and micropatterns on the outside to gain traction on the slippery bowel surfaces. For example, FIG. 6 is a diagram of a tread 600 with inner timing-belt teeth 602 and outer micropatterns 604. Continuity of the belt reduces traction/adhesion issues on the bowel surface, increases safety within the human body, and reduces wear of a noncontinuous belt that could delaminate or break from fatigue much sooner. The continuous belt allows for larger contact area on the colon mucosa, providing better traction and thus locomotion of the device, while still being a soft material that is safe for the colon tissue. The design of the OM/IM1/IM2 may allow for tunable thickness belts, depending on the application. Flexibility of the molded silicone (with negative micropatterns) allows easy peeling off without damaging delicate pillars, and the flexible mold can be reused. Use of a traditional mold for the negative pillars may separate in half or quarters from the inner mold, damaging pillars. Also, the PDMS material can be changed to any injectable and medically safe elastomer.

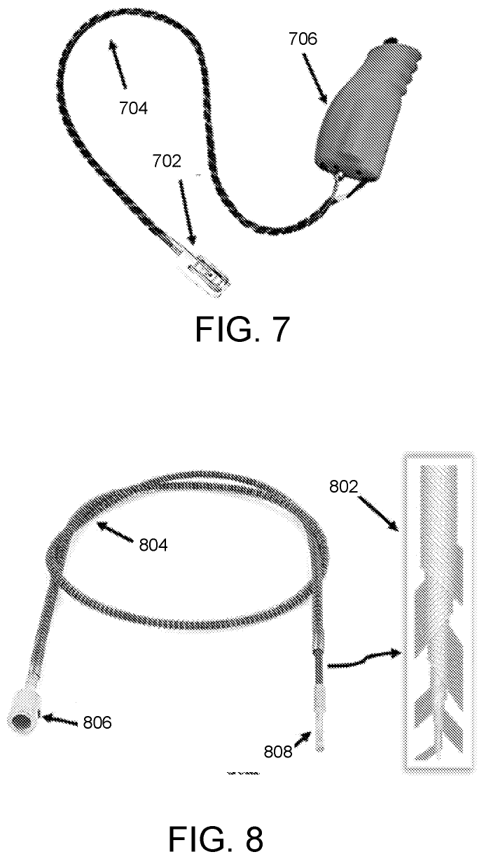

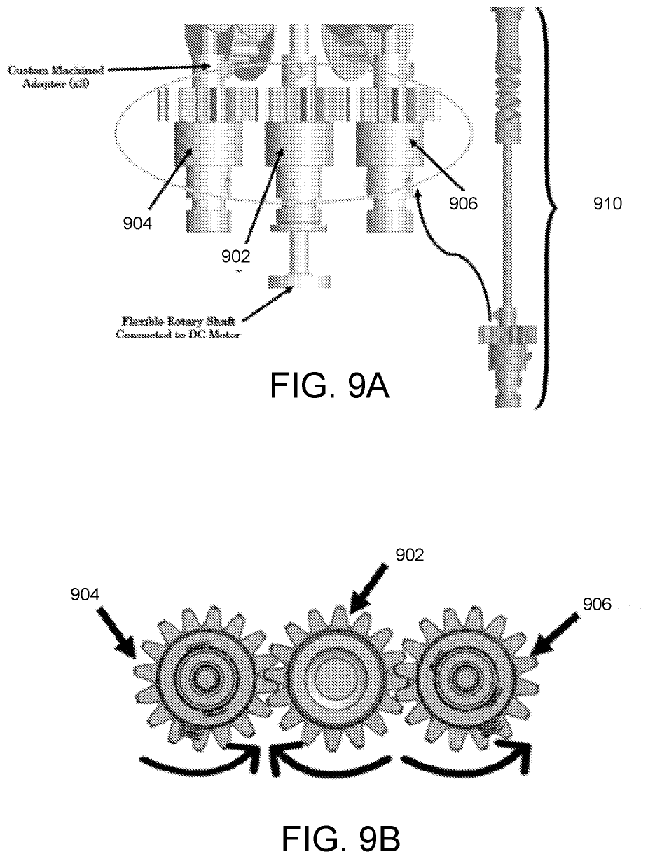

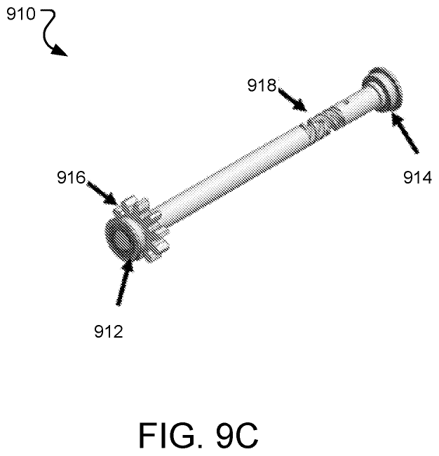

In some instances and as noted above, the RCE 100 may comprise the distal tip or end of an colonoscopy device powered through a flexible transmission system controlled by a handle. For example, FIG. 7 illustrates an isometric view of colonoscope including a distal tip 702 with a robotic capsule, a flexible tether 704, and a control handle 706. The components of the RCE 100 at the distal tip 702, particularly the rotary shafts of the robotic device, may be powered with a flexible power transmission connected to the handle 706 through the flexible cable 704. Thus, in this implementation, the robotic capsule 100 may not include onboard motors as described above. Instead, the worm support shafts of the RCE 100 may be powered by a flexible rotary shaft 900 (illustrated in FIGS. 9A & 9B) located within the RCE 100 and connecting the in vivo distal tip 702 and the ex vivo physician-operated handle 706. This flexible rotary shaft 900 transmits power and ultimately outputs locomotion at the distal tip 702. The flexible power transmission line 800 may be paired with a working channel, suction, and/or irrigation to enable a fully functional endoscope with active distal tip 702 that provide “pull” capability during use. Features may include the power transmission paired with treaded distal tip 702. In use, the shaft 900 and distal tip 702 would be single use, low-cost, while the handle 706 may be more expensive and multi-use. The use of the flexible robotic surgical system allows for minimally invasive surgery at a higher accuracy.

As described above, the double worm gear is included in the RCE 100. In general, a single worm can transmit power to multiple worm gears, and thus a single motor or rotary shaft can drive the top and bottom treads in the same rolling direction. If the worm gears are of the same right-handed spiraling and mirrored about the drivetrain, they produce a planar resultant force angled toward either top or bottom. The worm gear of the RCE 100 itself has both the right-hand and left-hand spiraling for both forward and reverse robotic motion. To get the device to propel in the same direction, an idler gear on the top geartrain may be included to regain similar rolling directions for both treads.

In one particular implementation, a flexible metal rotary shaft 802 may be utilized to deliver power from a motor off-board to the robot device 100 at the distal end 702 of the system. As illustrated in FIG. 8, the flexible rotary shaft 802 may be included in a sleeve 804 that connects the shaft from a handle using a handle motor shaft attachment 806 to the robot device 100 using a distal end attachment device 808. The flexible shaft 802 may be selected to provide a particular torque, directional operation, minimum bend radius, deflection, and speed. In general, the flexible shaft 802 may be made of layers of wires wrapped in a spiral formation. The number of layers, number of wires per layer, and size of the wires within each layer may all be selected as variables in flexible-shaft construction. In some instances, the diameter of the shaft 802 may directly impact the performance and capability of the rotary shaft in this particular implementation. In one example, the diameter of the flexible shaft 802 may be about 0.15″ (3.75 mm), although any diameter may be selected to meet the needs of propelling the robotic device 100.

The flexible rotary shaft 802 provides transmission of rotary motion through bends and curves of the flexible tether 704 without sacrificing efficiency. In particular, the flexible shaft 802 may located within the sleeve 804 which is connected between the RCE 100 and the handle 706 described above. Such a flexible rotary shaft may reduce dependency on alignment and may not require tight tolerances for uniform performance and complex maneuverability. By transferring power of the RCE 100 off-board for a pull CLS device opens the possibility to deliver a large range of power and torque to the robot and allows for the potential for a smaller, more compact distal tip 702.

The double worm gear configuration of the robotic device 100 described above allows for a custom shaft assembly which provides for off-board motorized power delivered directly from a middle driving gear. In particular, FIG. 9A illustrates an overhead view of a spur gear of the drive shaft of the robotic device 100 for transmitting power to the double worm gear. A center drive shaft 902 may be mechanically connected to the distal end of the flexible rotary shaft 802 at or near the distal end of the tether 704. The flexible rotary shaft 802 may translate rotation motion to the center drive shaft 902 to cause the center shaft to rotate. The rotation of the center drive shaft 902 may be transmitted to two parallel drive shafts 904, 906 through a gear drive train. In particular and as shown in FIG. 9B, rotation of the driving gear may cause corresponding rotation of a first drive shaft 904 and a second drive shaft 906. Rotation of the center drive shaft in a counter-clockwise direction causes rotation of the first parallel drive shaft 904 and the second parallel drive shaft 906 in a clockwise direction. Alternatively, rotation of the center drive shaft in a clockwise direction causes rotation of the first parallel drive shaft 904 and the second parallel drive shaft 906 in a counter-clockwise direction.

In one implementation, spur gears were selected to transmit power and rotation from the center drive shaft 902 to the parallel drive shafts 904, 906, although other types of gears may be used. The drive train may provide a reduced radial force acting on the gears with the input torque translated from the power transmission to the parallel drive shafts 904, 906. In this manner, the driving gear (middle gear) 902 may deliver equal speed and torque to the side driven gears 904, 906.

One or both of the parallel drive shafts 904, 906 may include a custom-designed drive shaft 910 with a double worm gear. In particular and as shown in FIG. 9C, the drive shaft 910 may include a proximal end 912 and a distal end 914. The proximal end 912 may include a spur gear 916 or other toothed gear. As described above, the spur gear 916 may be in mechanical communication with spur gears of the center drive shaft 902 to translate rotation of the center drive shaft to the parallel drive shafts 904, 906. The distal end 914 of the drive shaft 910 may include a flange at or near the distal end. In addition, a double worm gear section 918 may be located along the drive shaft 910 between the proximal end 912 and a distal end 914. The double worm gear section 918 may be configured similar to that described above to mechanically engage with the first worm gear 208 and/or the second worm gear 206 to drive or otherwise activate the gears of the robotic device 100. In this manner, the center drive shaft 902 may cause rotation of the worm gears of the robotic device 100 described above in place of including an on-board motor.

Through this implementation of the robotic endoscopic system, a single motor controls two microtextured treads moving simultaneously by the driven gears. The basis of the geartrain model allows for an introduction to the concept of powering the gear drive train off-board for pull via a flexible rotary shaft 802. In some instances, however, relying on a single motor limits the capability of manipulating the treads independently to allow for left and right actuation such that the robotic device 100 may be manipulated forward and rearward through the interaction of the flexible rotary shaft 802 with the central drive shaft 902 of the robotic device to activate the parallel drive shafts 904. 906 of the device drive train.

Several of the additional features discussed above may also be included in the robotic device 100 of this implementation. For example, a camera system could be integrated with the RCE 100 housing with an increase in the camera quality and improved resolution. In particular, a camera backend processor may be mounted inside or otherwise in communication with the handle 706. The handle 706 may activation buttons for various features of the camera, including a color balance button to adjust the lighting settings for inside the colon. A cord may connect the camera module of the RCE 100 to the processor of the handle 706 and included in the flexible tether cord 704. In one particular implementation, the camera cord may be wrapped in a spiral formation with the flexible rotary shaft 802. In addition, the cable for user visualization of the camera may connected from the processor in the handle 706 to an external computer screen.

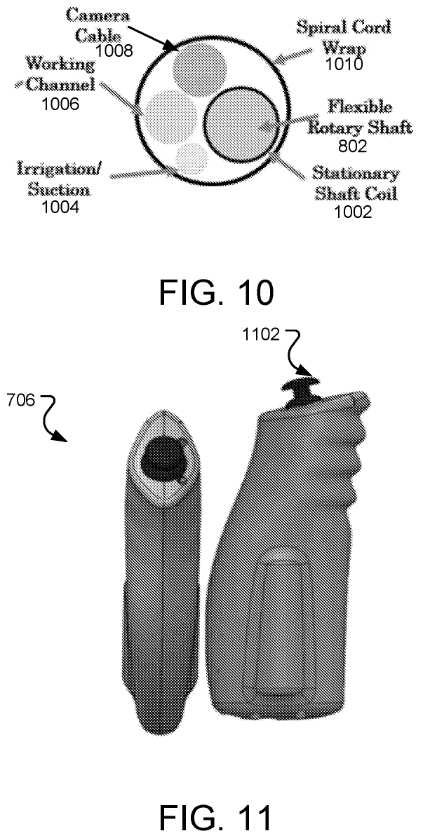

The flexible tether 704 may include cords for other features of the RCE 100 at the distal end 702 of the system. FIG. 10 illustrates a cross-section of the shaft coil for the system described herein. As shown, cords for various components of the RCE 100 may be wrapped in a flexible cord management system. The components included in the implementation shown are the flexible rotary shaft 802, camera cord 1008 for providing signals and power to a camera device, irrigation tube 1004 for delivering water and/or air through a port in the RCE, and an instrument port 1006. The tubes and cables of the tether 704 may be wrapped in the spiral cord management system 1002 that serve as a separation between the rotating components and an external sleeve 1010 for protection and low risk for cross-contamination. In general, the stationary coil 1002 may prevent the flexible shaft from damaging the camera cable 1008 and working channel 1006.

The handle 706 construction and, in particular, the DC motor torsional manipulation, provides a physician manual control over the RCE 100. The handle 706, as illustrated in FIG. 11, may include an analog joystick 1102 in communication with a microcontroller and DC motor driver for operating the RCE 100. The analog joystick 1102 allows for forward and reverse motion of the robot 100 and converts a reading from the internal potentiometer to a speed gradient output of the DC motor rotating the flexible shaft 802 to allow for precise motorized manipulation and direct control of the inputs to the robot. In general, the microcontroller of the handle 706 communicates with a motor driver board located within or outside of the handle to control the output of the motor and receives inputs from the analog joystick manipulated by the physician.

Operational Steps for Electromechanical Actuation of the system described herein is as follows:
1) Manipulate Joystick 1102 of handle 706 to provide an analog output to the microcontroller based on a potentiometer signal;
2) Clockwise or counter-clockwise rotational motion of the motor shaft within the handle occurs;
3) Corresponding rotation of the flexible shaft occurs;
4) Driving direction of the internal gears of the RCE 100 is transmitted through the gear train; and
5) Direction movement of the microtextured treads of the distal tip 702 locomotion.

Several components may be mounted internally within the handle 706, including but not limited to, a DC motor, stackable electronics, and a camera controller. The irrigation and suction components may be connected to its own machinery and will not necessarily be integrated with the handle assembly 706.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A robotic endoscope system comprising:
a central drive shaft comprising a first toothed gear and in mechanical communication with a flexible rotary shaft wherein rotation of the flexible rotary shaft corresponds to rotation of the central drive shaft and the first toothed gear in a first rotational direction;
a first parallel drive shaft comprising a second toothed gear in mechanical communication with the first toothed gear, wherein rotation of the first toothed gear in the first direction corresponds to a rotation of the first parallel drive shaft in a second rotational direction; and
a second parallel drive shaft comprising a third toothed gear in mechanical communication with the first toothed gear, wherein rotation of the first toothed gear in the first direction corresponds to a rotation of the second parallel drive shaft in the rotational second direction;
wherein the first parallel drive shaft and the second parallel drive shaft each further comprising a double worm section comprising a first screw section and a second screw section, wherein a spiraling component of the first screw section is different than a spiraling component of the second screw section; and
wherein the rotation of the first parallel drive shaft in the second rotational direction causes rotation of a first continuous track assembly and rotation of a second continuous track assembly.

2. The robotic endoscope system of claim 1 further comprising:
a first worm gear threadably engaged with the first screw section of the double worm section of the first parallel drive shaft and translating rotation of the first screw section of the first parallel drive shaft to a rotation of a first continuous track assembly; and
a second worm gear threadably engaged with the second screw section of the double worm section of the first parallel drive shaft and translating rotation of the second screw section to rotation of a second continuous track assembly.

3. The robotic endoscope system of claim 1 further comprising:
a third worm gear threadably engaged with the first screw section of the double worm section of the second parallel drive shaft and translating rotation of the first screw section of the second parallel drive shaft to a rotation of a third continuous track assembly; and
a fourth worm gear threadably engaged with the second screw section of the double worm section of the second parallel drive shaft and translating rotation of the second screw section to rotation of a fourth continuous track assembly.

4. The robotic endoscope system of claim 2 further comprising:
an idler gear threadably engaged with the second worm gear and the second continuous track assembly to transmit the rotation of the second screw section to the rotation of the second continuous track assembly.

5. The robotic endoscope system of claim 1 further comprising:
a housing connected to a distal end of the flexible rotary shaft, the housing encasing at least a portion of the central drive shaft, the first parallel drive shaft, and the second parallel drive shaft.

6. The robotic endoscope system of claim 5 further comprising:
a flexible tether connecting the housing at the distal end of the flexible rotary shaft to a control handle, the housing encasing at least a portion of the flexible rotary shaft.

7. The robotic endoscope system of claim 6 wherein the flexible rotary shaft translates a rotation movement of an actuator within the control handle to the central drive shaft through the flexible tether.

8. The robotic endoscope system of claim 6 wherein the flexible tether further comprises a camera control tube housing one or more control electrical cables in electrical communication with a camera within the housing.

9. The robotic endoscope system of claim 6 wherein the flexible tether further comprises an irrigation tube configured to deliver a liquid through an irrigation port of the housing.

10. The robotic endoscope system of claim 7 wherein the control handle comprises an input device in electrical communication with the actuator, wherein activation of the input device causes rotation of the flexible rotary shaft through the flexible tether.

11. The robotic endoscope system of claim 10 wherein the input device comprises a joystick input device.

12. The robotic endoscope system of claim 6 wherein the housing is located in vivo and the control handle is located ex vivo during a medical procedure.

13. The robotic endoscope system of claim 1 wherein the second continuous track assembly is angled with respect to the first continuous track assembly.

14. The robotic endoscope system of claim 1 wherein the first continuous track assembly and the second continuous track assembly both comprise a plurality of cylindrical treads extending perpendicular from a corresponding track surface.

15. A surgical method for a colonoscopy comprising:
locating a self-propelled colonoscope device in a gastro-intestinal tract of a subject;
mechanically transmitting, via a flexible rotary shaft in mechanical communication with a central drive shaft of the colonoscope device, a first drive force to rotate a first double worm drive shaft, wherein rotation of the first double worm drive shaft causes a first upper continuous track assembly to rotate in a first direction and a first lower continuous track assembly to rotate in a second direction opposite the first direction to propel the colonoscope device; and
mechanically transmitting, via the flexible rotary shaft, a second drive force to rotate a second double worm drive shaft independent of the first double worm drive shaft, wherein rotation of the second double worm drive shaft causes a second upper continuous track assembly to rotate independent of the first upper continuous track assembly and a second lower continuous track assembly to rotate independent of the first lower continuous track assembly,
wherein the colonoscope device comprises a flexible tether connecting a housing at a distal end of the flexible rotary shaft to a control handle, the housing encasing at least a portion of the flexible rotary shaft.

16. The surgical method of claim 15 wherein the flexible rotary shaft translates a rotation movement of an actuator within the control handle to the central drive shaft through the flexible tether.

17. The surgical method of claim 15 wherein the flexible tether further comprises a camera control tube housing one or more control electrical cables in electrical communication with a camera within the housing.

18. The surgical method of claim 15 wherein the flexible tether further comprises an irrigation tube configured to deliver a liquid through an irrigation port of the housing.

19. The surgical method of claim 16 wherein the control handle comprises an input device in electrical communication with the actuator, wherein activation of the input device causes rotation of the flexible rotary shaft through the flexible tether.

20. The surgical method of claim 15 wherein the housing is located in vivo and the control handle is located ex vivo during a medical procedure.
